(19) United States
(12) Patent Application Publication
(10) Pub. No.: US 2012/0203231 A1
(43) Pub. Date: Aug. 9, 2012

(54) FLEXIBLE MICRODRILLING INSTRUMENTATION, KITS AND METHODS

(75) Inventors: Marc Long, Denville, NJ (US); Kyle Craig Pilgeram, San Jose, CA (US); Marc Charles Wennogle, Centennial, CO (US)

(73) Assignee: HOWMEDICA OSTEONICS CORP., Mahwah, NJ (US)

(21) Appl. No.: 13/368,730

(22) Filed: Feb. 8, 2012

Related U.S. Application Data
(60) Provisional application No. 61/440,631, filed on Feb. 8, 2011.

(51) Int. Cl. A61B 17/16

(52) U.S. Cl. 606/80

(57) ABSTRACT

In one embodiment, the present invention may include a method of performing microdrilling surgery including directing a distal portion of a cannulated guide having an angle of curvature adjacent to a defect site; directing a drill, including a drill head and a flexible shaft, through the cannulated guide from a proximal portion of the guide through the distal portion and towards the defect site; drilling at least one hole into the defect site; and removing the drill and cannulated guide from the defect site and allowing blood or bone marrow to flow into the at least one hole towards the defect site.

### CROSS-REFERENCE TO RELATED APPLICATIONS

The present application claims the benefit of the filing date of U.S. Provisional Patent Application No. 61/440,631 filed Feb. 8, 2011, the disclosure of which is hereby incorporated herein by reference.

### BACKGROUND OF THE INVENTION

Microfracture surgery, also referred to as marrow stimulation, is generally an arthroscopic technique for the repair of a defect in articular cartilage. The procedure may also be performed as an open surgery. Typical microfracture repair includes the use of a pick or awl to puncture a series of small holes into subchondral bone. The pick or awl is typically used in a punching motion such as through the use of a hammer to punch the pick or awl into the bone to cause a fracture of the bone. The holes are intended to stimulate blood and bone marrow flow through the holes to the defect site. This flow usually results in what is commonly referred to as a clot or superclot.

Cartilage is naturally avascular and is unable to repair itself. Thus, the flow of blood and bone marrow, which may include stem cells, is intended to promote new cartilage growth.

Once the holes have been prepared, enhancement of the clot may be performed by the implantation of a material into the holes. Known materials include hydrogels, flowable matrices, porous scaffolds, membranes and tissues, cartilage fragments, and sealants and glues. These materials help promote good clotting of the blood and/or bone marrow.

Current microfracture techniques present several limitations. First, the use of the pick or awl may not ensure penetration of subchondral bone, to create a perforation to a proper depth, to maximize the flow of blood or marrow and to promote subsequent clotting. Second, the hammering motion of the pick or awl may crush the subchondral bone, which can inhibit the outcome of the procedure by crushing the porous structure of the bone surrounding the perforation. Third, subchondral bone sclerosis is often observed after these typical microfracture techniques. Fourth, the procedure may be time consuming. Fifth, the conical shape of the perforations due to the shape of the pick or awl is not optimal to access marrow or blood. Finally, some defects may be difficult to access using traditional microfracture instrumentation, particularly if the surgery is performed arthroscopically.

A recent development is the technique of microdrilling which is also intended to promote the repair of a soft tissue defect such as articular cartilage in the knee. In this technique, a drill is used to prepare bone holes, measured in millimeters, in the subchondral bone, or other bone underlying the soft tissue defect, by removing bone material and create channeling, to allow for the flow of blood, bone marrow, or both into the tissue defect area to stimulate regrowth of soft tissue, such as articular cartilage. FIG. 1 illustrates a recent study showing the differing results between these microfracture and microdrilling techniques. Chen, A Comparative Study of Microfracture and Drilling Surgical Techniques for Cartilage Repair, Poster No. 538, 54th Annual Meeting of the Orthopaedic Research Society. Microfracture (images c and f, right-hand side images) caused crushing of the porous bone structure resulting in minimal blood and bone marrow flow through the surrounding bone. Microdrilling (images b and e, center images) preserved the porosity of the bone resulting in ample blood and bone marrow flow through the bore hole.

However, the drill currently used in this technique is a straight drill, as is common in orthopedic surgery. This may limit the ability to reach some tissue defects within a joint. Additionally, even if the drill can access the tissue defect, it may not be capable of forming a bone hole which can reach the blood or bone marrow, e.g., the drill may not be capable of forming a bone hole which is generally perpendicular to the surface of the bone.

### BRIEF SUMMARY OF THE INVENTION

In a first embodiment, the present invention may include a device for performing microdrilling, also referred to microchanneling, surgery which may include a drill comprising a drill head and a flexible shaft, and a cannulated guide having an angle of curvature. The drill head may have a diameter adapted for creating bone holes in subchondral bone.

The flexible shaft may be constructed out of a solid tube constructed of nitinol, plastic, stainless steel or other surgically-acceptable material. The flexible shaft may be formed by either a series of laser cuts, which may or may not pass completely through the thickness of the shaft material, by the solid tube being constructed of flexible material, such as nitinol or plastic, or by any other suitable manner to establish some flexibility. Further, the laser cuts, if used to pass completely through the thickness of the shaft material, may form the tube into a series of interlocking portions or puzzle-piece portions. Alternatively, the laser cuts may form a spiral-cut configuration along at least a portion of the length of the tube to form the flexible shaft. Additionally, the flexible shaft may be capable of having an angle of curvature of between about 0 degrees to about 90 degrees, while still maintaining its function. For example, the angle of curvature may be about 0 degrees, about 45 degrees, or about 90 degrees. Alternatively, the flexible shaft may be capable of having an angle of curvature of between greater than 0 degrees up to about 90 degrees. For example, the angle of curvature may be about 15 degrees, about 30 degrees, about 45 degrees, or about 90 degrees. Though, the flexible shaft may be further capable of having a curvature angle up to about 180 degrees while still maintaining its function. The cannulated guide may include an angle of curvature sufficient to direct the drill head into the subchondral bone at a generally perpendicular angle to the adjacent bone surface.

Additionally, the cannulated guide may have a distal tip for engaging the defect site, such as the articular cartilage and/or the subchondral bone. In one embodiment, the distal tip may include a projection adapted to engage the defect site. For example, the projection may be a serrated edge. The distal tip of the guide may further include a window for viewing of the drill positioned within the guide. In another embodiment, the distal tip may include an offset pin which can engage the surface of the bone, an adjacent bone hole already prepared, or both. The offset pin may further be configured to provide proper spacing between bone holes at the defect site when placed within an already formed bone hole in preparation for forming another bone hole. In one arrangement, the offset may be retractable such that it can be retracted when drilling a first bone hole, but can then be extended for insertion into the first bone hole for the preparation of the second bone hole. Further, in some embodiments, the offset pin may be integral with or otherwise fixedly secured to the distal tip of the guide; may be rotatable, and lockable at various angles, about a circumference of the distal tip of the guide; or may be selectively removable from the distal tip of the guide.

In a further embodiment, the present invention may include a flexible drill including a drill head and a flexible shaft having an at least one wound coil. The flexible shaft may further include a plurality of wound coils positioned coaxial and concentric with one another. Each wound coil may include at least one filar, and may, for example, more specifically have 7 filars, 10 filars, 12 filars. The filars of a coil may be wound in a helical style such that each coil is essentially a multi-strand helix. Each wound coil may further include one of a clockwise turn or a counterclockwise turn, and the plurality of wound coils may include a portion of coils having a clockwise turn and a portion of coils having a counterclockwise turn. The plurality of coils, positioned axially and concentrically, may further include alternating coils of clockwise turn and counterclockwise turn. The flexible shaft may include five coils, or specifically layers of clockwise and counterclockwise multi-strand helixes wrapped on one another. Distal and proximal ends of each coil are secured to a drill head and a proximal portion of the drill, respectively. The proximal portion of the drill may be a fixed, linear shaft, and further a drill stop may be positioned at or adjacent to the securement point of the fixed and flexible shafts. The plurality of coils may be constructed of stainless steel or the like. The plurality of coils may be adapted to transfer torque from a surgeon, through the flexible shaft, and to the drill head, regardless of whether the drill is operating in a forward or a backward direction. The flexible shaft may be adapted to pass through a drill guide, or the like, having an angle of curvature between greater than 0 degrees up to about 90 degrees.

The present invention may further include a use of the above device for performing microdrilling surgery which may include a drill comprising a drill head and a flexible shaft, and a cannulated guide having an angle of curvature. The drill head may have a diameter adapted for creating bone holes in subchondral bone.

In another embodiment, the present invention may include a method of performing microdrilling surgery including directing a distal portion of a cannulated guide having an angle of curvature adjacent to a defect site; directing a drill, including a drill head and a flexible shaft, through the cannulated guide from a proximal portion of the guide through the distal portion and towards the defect site; drilling at least one hole into the defect site; and removing the drill and cannulated guide from the defect site and allowing blood or bone marrow to flow into the at least one hole towards the defect site.

The defect site may include subchondral bone, and further articular cartilage. The method may further include, prior to the step of directing the guide to the defect site, the step of debriding at least a portion of the articular cartilage. This additional step may, in some instances, expose subchondral bone. Further, the at least one hole may be generally perpendicular to a surface of the subchondral bone. The method may include the drilling of two or more holes into the defect site, by repeating the above steps of directing the distal portion of the guide the defect site, directing the drill to the defect site and drilling another hole into the defect site.

Further, the angle of curvature of the cannulated guide may be between about 0 degrees and about 90 degrees. For example, the angle of curvature may be about 0 degrees, about 45 degrees, or about 90 degrees. Alternatively, the flexible shaft may be capable of having an angle of curvature of between greater than 0 degrees up to about 90 degrees. For example, the angle of curvature may be about 15 degrees, about 30 degrees, about 45 degrees, or about 90 degrees. The distal portion of the cannulated guide may include a serrated edge, such that prior to drilling the hole into the defect site, the method may further include engaging the defect site with the serrated edge.

The method may further include the step of packing a material within the at least one hole following removal of the flexible drill. The material may pass through the cannulated guide to the bone hole or may be directed to the defect site through another instrument or entryway. The material may include a biomaterial, a scaffold, one or several growth factors, cells, cartilage particulates, cartilage matrix, a blood preparation, a bone marrow preparation, a tissue, or any combination thereof.

The method may further include another embodiment for drilling a series of holes into the subchondral bone. In this embodiment, the method may be performed using a cannulated guide having an offset pin at a distal tip of the guide. Using the offset pin, the pin may be placed into the first hole, which may provide proper spacing for placement of the second bore hole. This step may be repeated as necessary until substantially the entire defect site, and even adjacent area, may be covered by bore holes.

In a further embodiment, the present invention may include a method of performing microdrilling surgery including directing a distal portion of a cannulated guide having an at least one projection at a distal tip and an angle of curvature adjacent to a first location on a defect site; engaging the defect site with the projection; directing a drill, including a drill head and a flexible shaft, through the cannulated guide from a proximal portion of the guide through the distal portion and towards the defect site; drilling a first hole into the defect site; withdrawing the drill from the first bone hole; disengaging the projection from the defect site; and removing the drill and cannulated guide from the defect site and allowing blood or bone marrow to flow into the at least one hole towards the defect site. The method may further include, prior to the removing step, the additional steps of directing the distal portion of the cannulated guide to a second location on the defect site; engaging the defect site at the second location with the projection; drilling a second bone hole into the defect site; withdrawing the drill from the first bone hole; and disengaging the projection from the defect site. These steps may be repeated at least a third location to drill at least a third bone hole. The projection may include a serrated edge. The method may further include, prior to the step of directing the guide to the defect site, the step of debriding at least a portion of the articular cartilage.

In a further embodiment, the present invention may also include a method of providing instructions or information to practice any of the various methods of performing microdrilling surgery described herein. For example, the method may include supplying a surgical protocol, or like document, to provide step-by-step instructions for performing any of the method embodiments of the present invention.

In yet another embodiment, the present invention may include a kit for performing microdrilling surgery including at least one drill which may include a drill head and a flexible shaft; and a plurality of cannulated guides, each of the guides having an angle of curvature, a length, or both different from the others. The kit may further include a series of drills having different diameters than the others. The cannulated guides of the kit may have angles of curvatures from about 0 degrees to about 90 degrees. The kit may have additional cannulated guides having angles of up to about 180 degrees. For example, the various cannulated guides in the kit may have angles of curvature of 0 degrees, 45 degrees and 90 degrees; though alternative steps of curvature between guides is also envisioned, such as a kit having guides with an angle of curvature every 10 degrees from 0 degrees to 90 degrees, or the like. Alternatively, the kit may include cannulated guides having an angle of curvature of between greater than 0 degrees up to about 90 degrees. For example, the angle of curvature of the various guides in the kit may be about 15 degrees, about 30 degrees, about 45 degrees, about 60 degrees, and about 90 degrees.

The kit may further include cannulated guides having an assortment of distal tips, such that the surgeon may select the proper distal tip for the location of the tissue defect. In one embodiment, the cannulated guides may all include offset pins. The offset pin on each guide may be positioned on a different side of the guide, relative to the curvature of the guide. Alternatively, some or all of the cannulated guides may include a distal tip having more than one offset pin on a single cannulated guide, wherein each offset pin is at an angle relative to the other offset pin or pins on the cannulated guide. In yet another alternative, the kit may include a plurality of offset pins which may be selectively attached to a distal tip of a receiving guide. The various offset pins may have various offset distances and surfaces which may be at various angles.

The kit may further include an instrument for implanting a material into a hole prepared by the drill within a patient on which the kit was used.

In yet another embodiment, the method include the preliminary step of preparing the cartilage defect by removing some of the damaged cartilage and shaping the defect to expose the calcified cartilage or the subchondral bone using curettes and shaping tools. The aforementioned kit may then also include a set of curettes and shaping tools.

### BRIEF DESCRIPTION OF THE DRAWINGS

FIG. 1 illustrates cross-sectioned tissue following both a microdrilling procedure (“MD2” in images a and d, as well as images b and e) and a microfracture procedure (“MF2” in images a and d, as well as images c and f).

FIGS. 2-5 illustrate various embodiments of drill heads on a flexible drill of the present invention.

FIGS. 6-10a-d illustrate various embodiments of flexible shafts on a flexible drill of the present invention.

FIG. 11 illustrates one embodiment of a cannulated guide of the present invention.

FIGS. 12-13 illustrate various embodiments of distal tips for a cannulated guide of the present invention.

FIGS. 14, 15a-c, 16a-b, and 17a-d illustrate various embodiments of offset pins on distal tips of cannulated guides of the present invention.

FIG. 18 illustrates one embodiment of a device, illustrating one arrangement of a cannulated guide and a drill, of the present invention.

FIGS. 19-20a-c illustrate further embodiments of a cannulated guide of the present invention.

FIGS. 21-23 illustrate one embodiment of a method of microdrilling of the present invention.

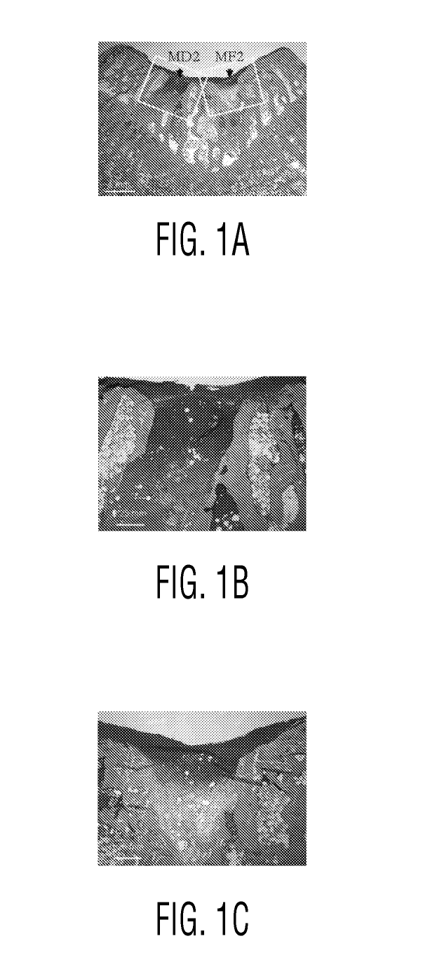

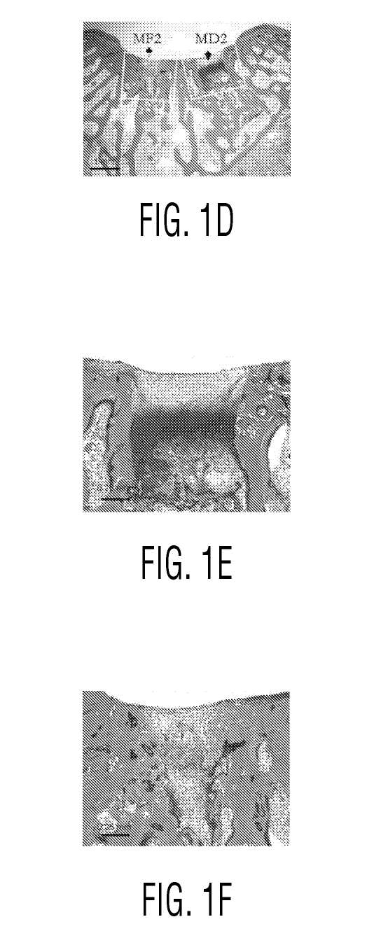

### DETAILED DESCRIPTION

For simplicity, the various devices, methods and other embodiments disclosed herein will pertain to the repair of a defect site in articular cartilage in a knee by using the drill and related instrumentation on the articular cartilage (if present) at the defect site and subchondral bone below and adjacent to the articular cartilage defect, though this invention may be used in other joints or other areas of the body for the treatment of other avascular tissues which may be located in the hip, shoulder, talus or ankle, wrist, elbow, digits, spine, or other joints within the body.

While the embodiments are described for human joints, this invention may be used in other species, including horses and dogs for example, though in the example of dogs, the instrumentation will likely be smaller than those used on horses and humans.

A first embodiment of the present invention includes instrumentation for microdrilling designed to drill subchondral bone to assist in repair of the soft tissue defect. The instrumentation includes a flexible drill 10 and at least one cannulated guide 50 through which the flexible drill may pass. The guide 50 or guides may be straight or curved such that they are capable of accessing the defect site within a patient.

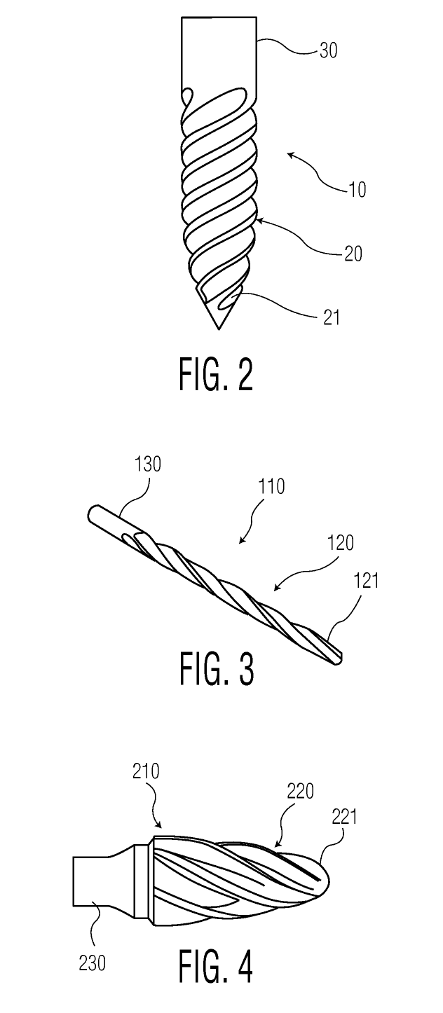

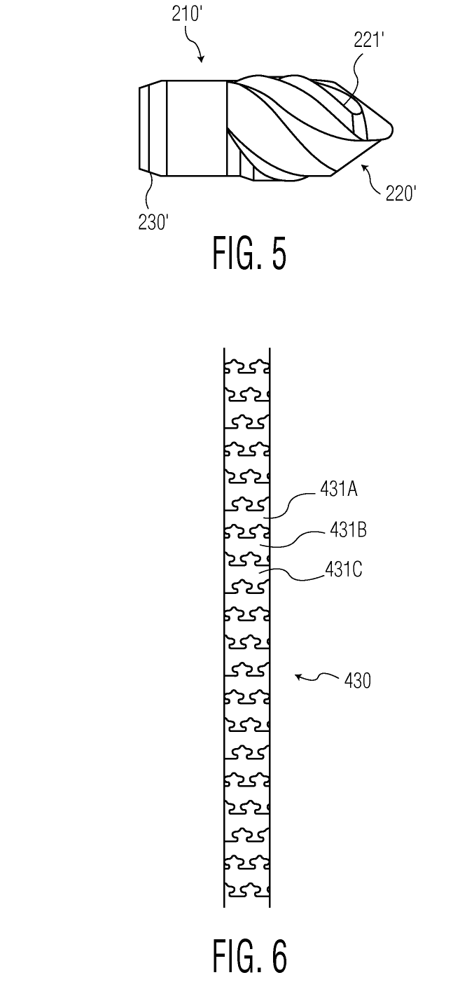

The flexible drill 10, 110, 210, 210′, 310 includes a drill head 20, 120, 220, 220′, 320, as illustrated in FIGS. 2-5 and 10A, for example. The drill head 20, 120, 220, 220′, 320 may have a diameter, at a widest point, of about 0.5 mm to about 5 mm, though some embodiments may be between about 1 mm and about 3 mm, and yet further embodiments may be between about 1.5 mm and about 2.5 mm. Preferably, the diameter of the drill head is about 2 mm, resulting in a bone hole of about 2 mm. The drill head 20, 120, 220, 220′, 320 may have a length sufficient to drill into subchondral bone to a depth of between about 1 mm and about 20 mm, though some embodiments may be between about 3 mm and about 15 mm, while further embodiments may be between about 3 mm and about 10 mm. An alternative drill head length may be sufficient to drill a hole into the subchondral bone to a depth of between about 3 mm to about 7 mm into the bone.

The shape of the drill head 20, 120, 220, 220′, 320 may be substantially cylindrical and may include at least one flute 21, 121, 221, 221′. If multiple flutes 21, 121, 221, 221′ are present, the flutes may be substantially symmetrical or at least one flute may be larger than the others. The head may, in another arrangement be a generally conical shape, such that at least a portion of the head includes a taper. In another alternative arrangement, the drill head may instead be a burr tip, as are known in the art. It may thus be substantially spherical in shape and shorter in length than a substantially cylindrical head. This may allow for increased clearance through a cannulated guide having a large angle of curvature or bend, which will be discussed in greater detail below. In yet another arrangement, the tip may be a trocar tip 420, as in FIG. 8, which may function as a drill, i.e., rotate about its central axis, to prepare the bone hole.

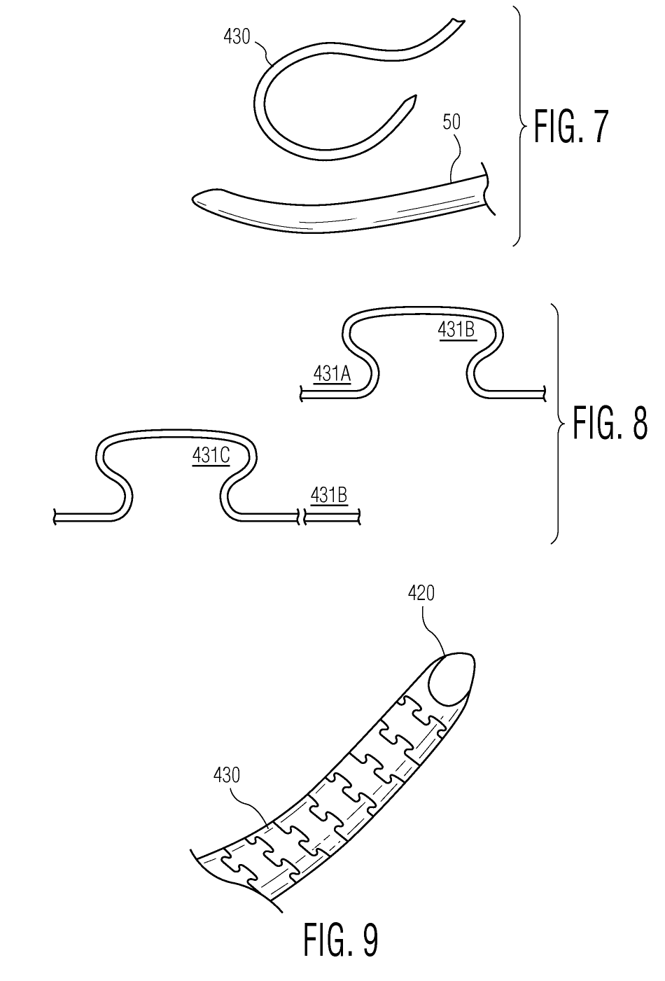

The flexible drill 10, 110, 210, 210′, 310 may also include a flexible shaft 30, 130, 230, 230′, 330, 430. The purpose of the flexible shaft is to allow the drill to pass through a cannulated guide, having a curved portion along its length, while remaining functional. In one embodiment, the flexible shaft 430 may have a series of interlocking portions 431a, 431b, 431c (and so on), such as a puzzle-piece configuration, as are illustrated in FIGS. 6-9. In this embodiment, the flexible shaft may start as a solid tube of material, which is then laser cut circumferentially to form the interlocking cuts. The cuts may be of any pattern, though an interlocking pattern is illustrated in FIGS. 6-9. Examples of such flexible shafts are also disclosed in co-pending U.S. patent application Ser. No. 12/460,310 (“Suture Anchor Implantation Instrumentation System” filed Jul. 16, 2009); Ser. No. 12/821,504 (“Suture Anchor Implantation Instrumentation System” filed Jun. 23, 2010); and Ser. No. 12/859,580 (“Flexible ACL Instrumentation, Kit and Method” filed Aug. 19, 2010), each of which is incorporated by reference herein as if fully set forth herein.

Alternatively, the cuts may pass through only a portion of the depth of material, such that the portions, between the laser cuts, are not discrete from one another. In this configuration, an interlocking pattern may not be necessary, and thus a sinusoidal, linear, spiral, or other circumferential pattern may be used instead. The solid tube may be any biologically acceptable material for use in surgery such as, for example, nitinol, plastic, stainless steel, hypodermic metal tubing, or the like. Some materials, such as nitinol or plastic, may not require deep laser cuts, or may not require laser cuts altogether, but may instead be designed to provide adequate bending capability for passage of the drill through a curve in the cannulated guide while maintaining the functionality of the drill instrumentation without the use of laser cuts.

In another embodiment, a flexible shaft may have a spiral-cut configuration (not shown), as discussed above. In this embodiment, the flexible shaft includes a spiral cut along at least a portion of the shaft. Such a spiral cut may be only partially through the depth of the shaft, such that the cut may impart flexibility, but the shaft remains a solid tube of material. Alternatively, the spiral cut may pass completely through the depth of the shaft. Where the spiral cut passes completely through the depth of the shaft, the shaft may be held together (e.g., to prevent unwinding during backwards drilling, such as when backing the drill out of the defect site) by a wire or the like passing through the length of the shaft, and attaching to the ends of the flexible shaft, which maintains a desired length between the two ends of the shaft.

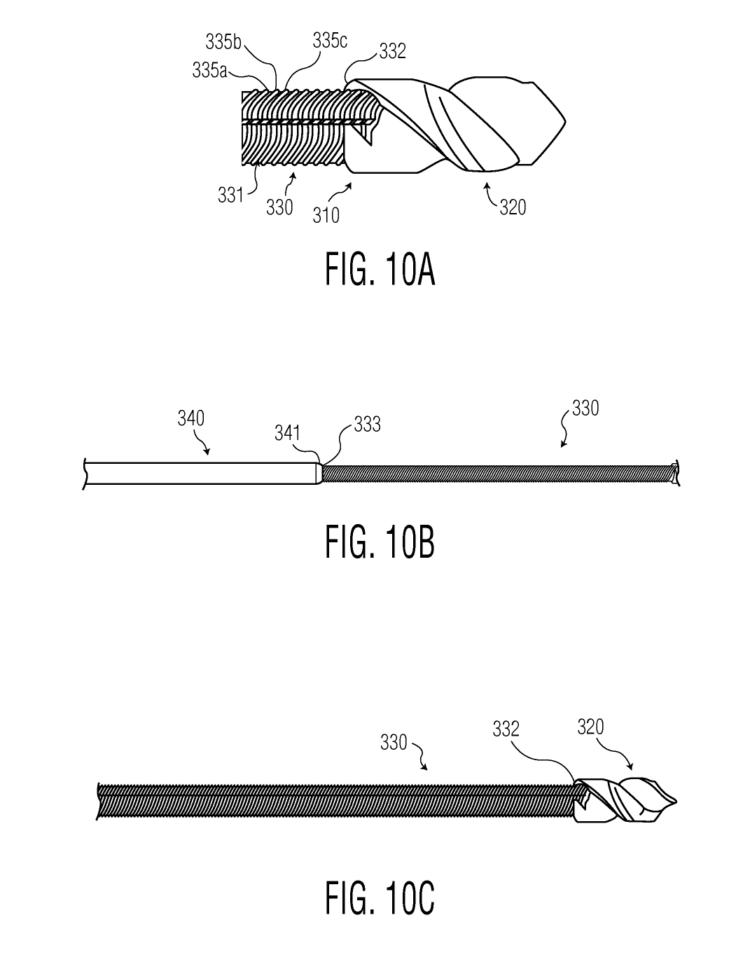

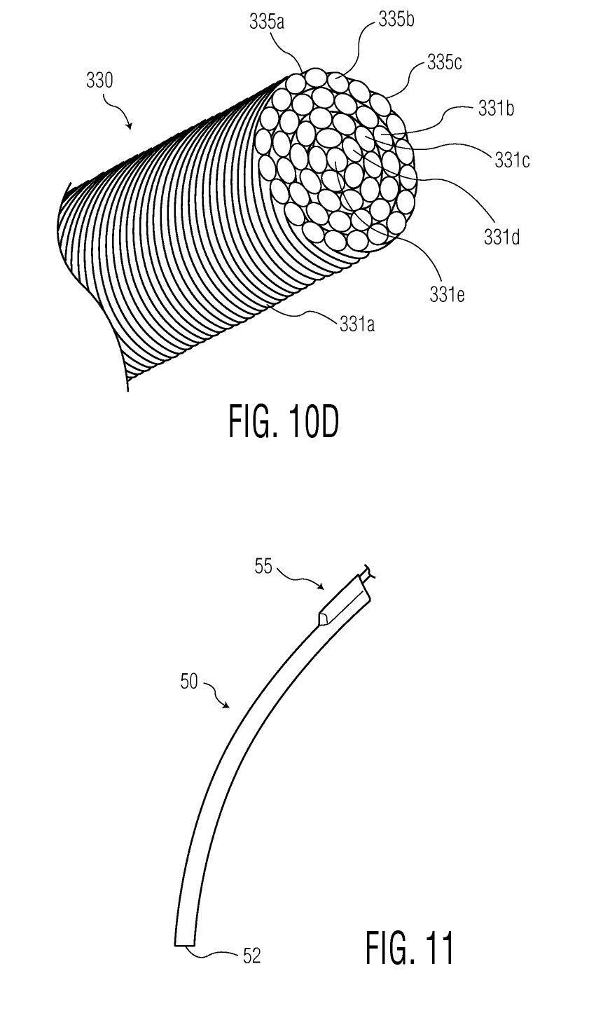

In a further embodiment, illustrated in FIGS. 10A-D, a flexible shaft 330 may have a spiral configuration in the form of an at least one wound coil 331. The ends of coil 331, including distal end 332 and a proximal end 333, may be welded, or otherwise secured, to the drill head 320 and to a distal end 341 of a fixed, linear shaft 340 or like structure, respectively (see FIGS. 10A, 10B and 10C). The coil 331 may be spring-like, and like a spring, may have compression and expansion properties. The coil is constructed from, for example, a plurality of wound stainless steel filars 335, or the like, which are twisted to form a multi-strand helical structure. As illustrated in FIGS. 10a and 10d, filars 335a, 335b, 335c (and so on) are wound around a central bore in a helical fashion to form coil 331 of flexible shaft 330. Such a flexible shaft, generally speaking, may be, for example, HHS® Tube (Fort Wayne Metals, Inc., Fort Wayne, Ind.).

Further, the flexible shaft 330 of this embodiment may include more than one coil 331. As illustrated in cross-section in FIG. 10D, for example, the flexible shaft may include a plurality of coils 331a, 331b, 331c, 331d, 331e configured to be coaxial and concentric with one another. Thus, within the circumference formed by outer coil 331a, may be at least one more similarly shaped coil of a smaller diameter spiral than coil 331a, in effect forming concentric rings of increasingly smaller diameter coils. Each coil may include at least one filar 335. In the illustrated embodiment, for example, coil 331a may include 12 filars 335, coil 331b may include 12 filars 335, coil 331c may include 10 filars 335, coil 331d may include 7 filars, and coil 331e may include 1 filar. The flexible shaft may, overall, include between about 20 to about 50 filars, forming a structure of concentric rings of coil from the largest, outer coil 331a to a much smaller, innermost coil 331e. Of course, an alternative number of filars, above 50 or below 20, is also envisioned. Each coil may be individually welded, or otherwise secured, at its proximal and distal ends, to the fixed shaft 340 and drill head 320, respectively. Of course, any number of coils may be used depending on the anticipated application of the drill, the strength of tissue through which the drill is to pass, and the anticipated size of bore hole to be prepared.

In the embodiment of a flexible shaft having multiple coils positioned concentrically and coaxially with one another, as in FIGS. 10A-D, each coil may have one of a clockwise turn or a counterclockwise turn, so long as at least one coil has a clockwise turn and at least one coil has a counterclockwise turn. For example, coil 331 may have a counterclockwise turn such that, upon drilling into the defect site (with the shaft spinning in the clockwise direction), coil 331 compresses and transfers the torque from the surgeon and fixed shaft 340 to the drill head 320 to promote drilling. When removal of the drill is necessary, and thus the drill would be operated in reverse (with the shaft spinning in the counterclockwise direction), the second coil 331b, for example, compresses and transfers the torque from the surgeon and the fixed shaft 340 to the drill head 320 to allow for removal of the drill. However, if only coil 331 were present, then upon operation of the drill in reverse, the coil 331 may unwind due to the torque created by the surgeon since the shaft 330, 340 is spinning in the same direction as the turn of the coil 331a while the drill head 320 is lodged in the defect site. The second coil 331b, including the opposite turn (i.e., a clockwise turn) of coil 331a, prevents such unwinding. Thus, in a flexible shaft having two coils of opposing turns, one of the coils acts to compress and transfer the torque from the surgeon, through the flexible shaft, and to the drill head regardless of whether the drill is operating in a forward rotation or a reverse rotation. In the example of the flexible shaft including the five coils 331a, 331b, 331c, 331d, 331e, three of the coils (331a, 331c, 331e) have a counterclockwise turn while the other two coils (331b, 331d) have a clockwise turn. In a preferred embodiment, there may be at least one additional coil having the counterclockwise turn than coils having a clockwise turn, which may provide greater torque for the drill to operate in the forward, or drilling, direction. Further, it is preferred to have alternating counterclockwise coils and clockwise coils positioned concentrically within the flexible shaft, as in the illustrated embodiment of FIG. 10D. However, of course, any number of coils having either a clockwise or counterclockwise turn is envisioned.

In some embodiments, such as in FIGS. 2-5, 7 and 9, the flexible shaft and drill head may be secured to one another to form a monolithic structure. In one arrangement, the shaft and head may be formed from the same piece of material, such that the entire structure is shaped from that single piece of material, such as nitinol. Similarly, the entire flexible drill may be constructed of stainless steel, such that the drill head and interlocking portions may be constructed from the single piece of material. Alternatively, a nitinol or plastic shaft may be welded or otherwise bonded to a stainless steel drill head to form a single structure. Other combinations may also be used, so long as the flexibility of the shaft and the strength of the overall structure are achieved.

Regardless of the embodiment of flexible shaft used, the flexible shaft may be capable of achieving a curvature of between about 0 degrees to about 90 degrees, though a curvature angle of greater than 90 degrees may also be achieved, while still maintaining the function of the drill, such as, for example, an angle of curvature up to about 180 degrees. Alternatively, the flexible shaft may be capable of having an angle of curvature of between greater than 0 degrees up to about 90 degrees. For example, the angle of curvature may be about 15 degrees, about 30 degrees, about 45 degrees, or about 90 degrees. In one embodiment, the angle of curvature of the flexible drill may be between about 30 degrees and about 70 degrees, while a further embodiment may have an angle of curvature between about 45 degrees and 60 degrees. The curvature radius may be about 20 mm or less, in order to navigate the tight confines of a joint and reach a defect site anywhere within the joint. The curvature radius may be adjusted by various parameters related to the flexible shaft. For example, the curvature radius may be adjusted by preparing the gap distance between interlocking portions in the puzzle-piece embodiment of the flexible shaft, as with shaft 430. Thus, if the gap between two interlocking portions is 0.025 mm, the curvature radius will be about 37.5 mm. However, if the gap distance is expanded to 0.05 mm, the curvature radius decreases to about 20 mm because adjacent interlocking portions have greater movement relative to one another with the larger gap distance between them. Further, if the gap distance is expanded to 0.064 mm, the curvature radius decreases to 15 mm. The increase of the gap distance allows for greater movement between adjacent interlocking portions, which may provide for an increase in directional change between interlocking portions while minimizing any increase in stress between adjacent interlocking portions resulting from the curvature obtained.

The flexible shaft 330 of FIGS. 10A-D may have a tighter radius of curvature, for example, less than about 10 mm. Such a small radius of curvature may be a result of the construction of the layers helixes and coiled strands.

The various cannulated guides disclosed herein, which include an angle of curvature, may have a radius of curvature of about 10 mm. Thus, the various flexible shafts disclosed herein may be dimensioned to accommodate such a radius of curvature.

As illustrated in FIG. 7, the shaft 430 may be capable of forming a “U”-shape if the gap distance is large enough to accommodate such flexibility, and thus have a curvature angle of about 180 degrees, though, as suggested in FIG. 7, the flexible shaft may be capable or forming a complete circle and thus having a curvature angle of 360 degrees.

In one embodiment, illustrated in FIG. 6-9, one example of the flexible drill may include a stainless steel shaft 430 including laser cuts which form discrete interlocking portions 431a, 431b, 431c, or puzzle-piece segments, and a trocar tip 420 which operates as a drill head which is welded to the end of the flexible shaft. In this example, the dimensions of the trocar tip are such that the distance from the extreme tip of the trocar to the first puzzle-piece cut is about 8.3 mm, which is a length which may create a bone hole of sufficient depth to achieve the flow of blood, bone marrow or both into the defect site through the bone hole. The flexible shaft of this embodiment may have an outer diameter of about 2.3 mm.

The flexible drill should be capable of operating at a drilling speed sufficient to create a hole in subchondral tissue, while maintaining a temperature below that which may cause necrosis of the bone adjacent to the hole being drilled. For example, the temperature of the surrounding tissue should remain at or near normal body temperature—about 37 degrees Celsius—throughout the drilling process, though allowing the temperature to increase slightly to under 40 degrees Celsius, if for only a short period of time, is also sufficient. In other examples, allowing the temperature to increase to 50 degrees Celsius, or even to 60 degrees Celsius, for a short period of time may also be sufficient and may not cause necrosis so long as the time the tissue undergoes such a temperature change is minimal. The drill speed should be sufficient such that it may cleanly prepare a hole in the bone by removing and not compacting the bone material while minimizing any crushing of the porous structure of the adjacent bone. Such clean preparation maximizes the probability of success of surgery because the porous structure of the bone around the drill hole remains intact to allow for proper flow of nutrients, blood or bone marrow, or any combination of these, to the defect site. In one example, the drill may operate at a speed of about 1500-1600 RPM.

The flexible drill may also have one or more depth stops to inhibit the movement of the drill into the bone at a certain, pre-determined depth. The depth stop is designed such that the drill may penetrate the subchondral bone deep enough to reach the vascular channels providing the marrow as well as the source of cells and blood components. This depth may vary between humans—and also between species—based on the bone quality, sometimes linked to the progress of an underlying disease, such as osteoarthritis or osteoporosis, and other factors. The depth may also vary based on the anatomy at the defect site or the specific joint in which the procedure will take place. The depth stop may prevent a surgeon from advancing the drill too far into the subchondral bone, which may cause significant damage to the underlying bone. For example, the drill should not penetrate too deep to reach the marrow cavity, such that the depth stop would typically halt the advance of the drill head once a depth of about 5-6 mm below the calcified cartilage layer that delineates the subchondral bone has been obtained. Any depth further than this may cause damage to the bone and, moreover, may be unnecessary to achieve the desired results of this procedure.

In one embodiment, the depth stop may be a flat face, perpendicular to the longitudinal axis of the drill, which abuts the surface of the subchondral bone, or alternatively a matching structure on the cannulated guide. In the alternative, the depth stop may also be on the opposite end of the drill shaft, which abuts the entryway of the cannulated guide. In another embodiment, the depth stop may be set at a distance such that the puzzle-piece portions of the flexible drill are prevented from exiting the cannulated guide and entering into the defect site or underlying bone. Of course, consideration of a proper length drill head must be performed prior to use to ensure the drill will still reach a sufficient depth into the bone. For example, in a further embodiment, the drill and guide (or a kit including same) may include a series of stops, such as movable washers or the like, which may be positioned on the shaft by the surgeon such that the surgeon can individualize the stop (and thereby, the drill depth), or plurality of stops, for a specific anatomy, patient, or procedure.

In yet another embodiment, one example of a depth stop is illustrated in FIG. 10c, wherein the distal end 341 of the fixed shaft 340 includes a shoulder formed by the securement of the smaller-diameter flexible shaft 330 to the larger-diameter fixed shaft 340. A cannulated guide (not shown) may have an inner diameter substantially equal to the diameter of the flexible shaft, but smaller than the diameter of the fixed shaft, or at least a portion of the fixed shaft, such that the portion of the fixed shaft cannot enter into the proximal end of the guide.

Furthermore, this embodiment may also include a cap (not shown) capable of securing to the proximal end of the guide, such that the cap allows the surgeon to adjust the drill depth according to the size of the cap secured to the proximal end of the guide. The cap may be sized such that the diameter of the fixed shaft cannot enter into the cap. For example, a cannulated guide, or alternatively, a kit including a plurality of cannulated guides, may also include at least one cap which may be interchangeable with the guide or guides and may allow the surgeon to adjust the drill depth. In this example, a guide or a kit may include at least two caps, one having a length of 4 mm, and one having a length of 5 mm, such that the surgeon can shorten the drill depth by 4 mm or 5 mm, respectively.

In addition to, or separate from, the cap or caps, the handle of the cannulated guide, such as handle 952 of FIG. 19, may have a threaded engagement with the guide tube 951 such that the overall length of the cannulated guide may be adjusted to thereby adjust the hard stop (i.e., the proximal end of the handle) relative to the distal tip of the guide. In one example, this threaded engagement may be used to fine tune to the drill depth once a cap, or other hard stop, is positioned on the instrumentation.

The instrumentation also includes at least one cannulated guide 50, such as the example illustrated in FIG. 11, which may include a cannulated guide 52 capable of receiving the flexible drill and directing the drill head to a defect site in a joint. The aforementioned pending patent applications, incorporated by reference herein, likewise include examples of such cannulated guides. The cannulated guide may include a curvature between about 0 degrees and about 90 degrees, and may match the curvature parameters of the flexible drill to which it is matched. In one embodiment, the instrumentation may include a plurality of cannulated guides, each having a different degree of curvature than the others, ranging from 0 degrees to 90 degrees, progressing in 5, 10 or 15 degree increments, though other increments may be used for specific applications. Examples of possible curvature angles in a set of cannulated guides may include 45 degrees, 60 degrees and 90 degrees. In an alternative example, a set including cannulated guides may include angles of curvature of 0 degrees, 45 degrees and 90 degrees. Alternatively, the cannulated guides may have an angle of curvature of between greater than 0 degrees up to about 90 degrees. For example, the angle of curvature of the various guides may be about 15 degrees, about 30 degrees, about 45 degrees, and about 90 degrees. Of course, if required for a particular surgical procedure, a cannulated guide may have an angle of curvature of more than 90 degrees to about 180 degrees, such that it is U-shaped.

In one embodiment, the guides may be selected such that the bone holes are drilled substantially perpendicular to the articulating surface. Drilling subchondral bone at an angle not substantially perpendicular to the articulating surface may weaken the bone structure and induce collapse of subchondral bone. In situations where the subchondral bone is healthy and strong, such as in young patients, this may not be an issue. However, in situations where the subchondral bone is weakened by an underlying disease or injury or from advanced age of the patient, drilling at an angle not substantially perpendicular to the articulating surface may result in bone collapse, and thus, the appropriately angled guide should be selected to access the defect site and to drill substantially perpendicular to the articulating surface. For these reasons, the instrumentation may provide for options for the cannulated guide which will provide drilling substantially perpendicular to the articular surface no matter where the defect is located within the joint or what angle of entry the instrumentation approaches the defect.

The structure of the cannulated guide may include a wall thickness of about 0.5 mm to about 1 mm, and preferably about 1 mm. The wall thickness should be as thin as possible to minimize the overall size of the instrumentation, while having sufficient thickness to prevent bending of the guide by use of the flexible drill. The cannulated guides may be constructed out of stainless steel, plastic, hypodermic metal tubing, or other material.

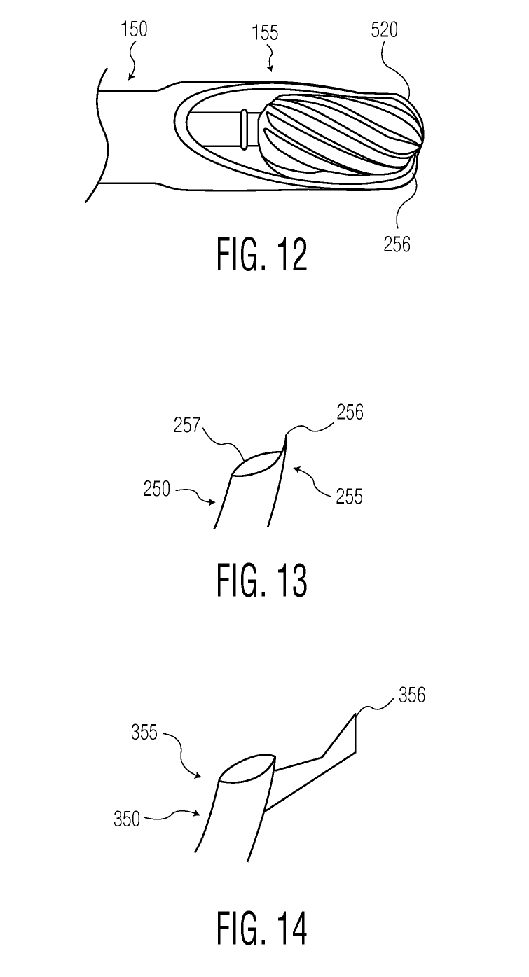

The cannulated guide 50 includes a distal tip 55 which may be designed to be positioned at the defect site. Various structures positioned on the distal tip are illustrated in FIGS. 11-20. The tip may further be designed to seat against the surface of the subchondral bone, a calcified cartilage layer, or articular cartilage, if any is present, or any layer present which at the time of the surgery forms the end of the bone structure within the articulating joint. In one embodiment, illustrated in FIG. 12, the distal tip 155 may be an angled cut. The angled cut may assist the drill head 520 in exiting from the guide 150, particularly if the guide has a large degree of curvature. The angle may also assist in visualization of the drill head as it exits from the guide. The distal-most portion 256 of the angled cut may also be placed directly onto the tissue surface, at the defect site, to provide a stable support for drilling the bone hole. In another embodiment, illustrated in FIG. 13, the distal tip 255 may include a projection, such as spike 256, on the rim 257 of the distal tip which may contact the tissue surface which may provide a stable support for drilling the bone hole and may further dig into the subchondral bone surface for added stability. Yet another embodiment may include a distal tip having a clear portion such that the drill head can be visualized as it approaches the distal tip and, thus, the defect site on the tissue. The clear portion can either be a small window on at least a portion of the circumferential surface of the distal tip of the guide or substantially the entirety of the distal tip of the guide may be constructed of a clear material for full visualization from any angle.

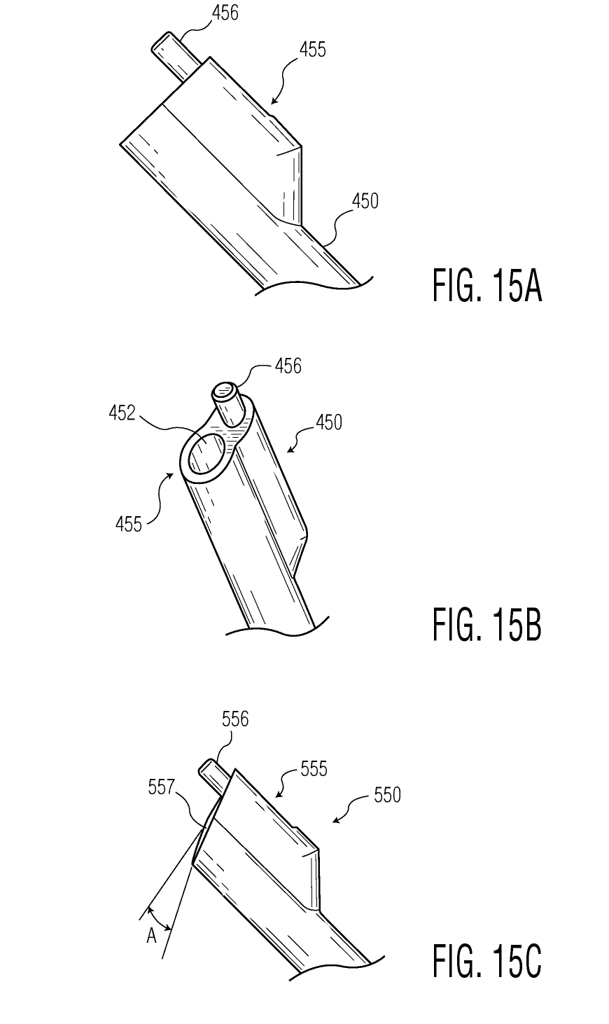

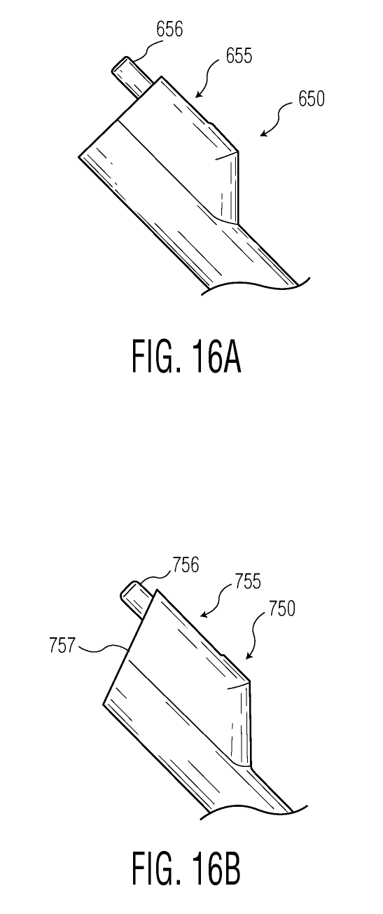

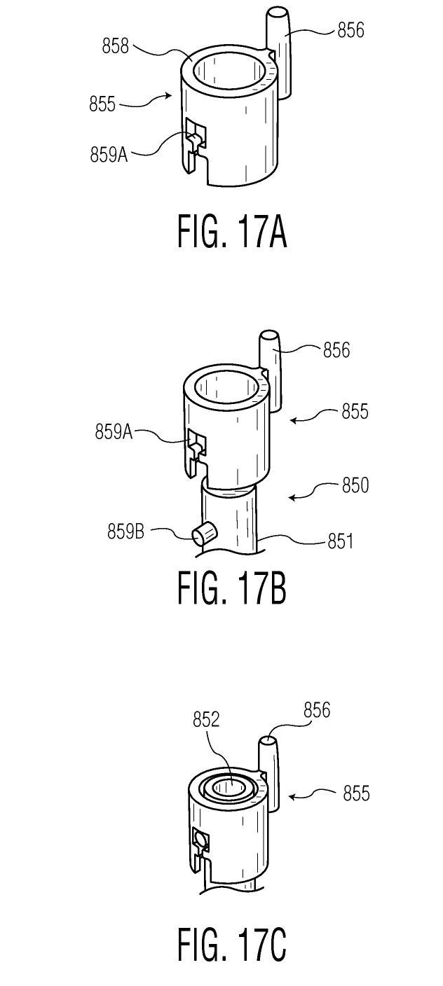

A further embodiment of the distal tip of the guide, illustrated in FIGS. 11, and 14-18, may include a distal tip 355, 455, 555, 655, 755, 855 having an offset pin 356, 456, 556, 656, 756, 856 which may, for example, provide a stable support for drilling by contacting the tissue surface. These embodiments may have a further feature in that, once the first bone hole is drilled, the offset pin may be placed within that bone hole and a second bone hole may be prepared adjacent to the first bone hole at a distance specified by the degree or distance of offset of the pin. This procedure may be repeated as needed. The offset pin may be configured to be a specified offset distance from the axis of the cannulated guide, for example, about 3 mm, as illustrated in FIGS. 15a-c. This distance may, however, be any distance desired, such as for example, in FIGS. 16a-b, an offset distance of about 4 mm may be used. The offset distance may provide for properly distanced bone holes to provide for maximized blood and bone marrow flow to the defect site. This distance may be designated by the surgeon based on patient demographics, subchondral bone density, or other similar factors to ensure proper spacing of the bone holes, and such designation may be used in manufacturing the cannulated guides for use by that particular surgeon. Further, the pin may be spring-loaded and thus retractable such that it is not used when the first bone hole is prepared (and thus the distal tip of the guide rests directly on the defect site or subchondral bone or other tissue). Then, once a first bone hole is in place, the pin may be extended and placed within the first bone hole. Of course, the pin may also be retracted if the surgeon is preparing a bone hole without concern for its relative distance from already prepared bone holes.

For example, in one embodiment, the drill diameter may be about 2 mm and the pin diameter may be about 1.5 mm. The axis of the pin is offset about 3 mm from the axis of the drill. Thus, when the pin is placed within an already prepared bone hole, the pin would be forced against the side of the bone hole, since the pin is smaller than the drill, and thus the axis of the second bone hole would be positioned about 3.25 mm from the axis of the first bone hole. Such a configuration would provide for at least about 1.25 mm of bone between the walls of adjacent bone holes, which may provide for sufficient strength to maintain the integrity of the bone holes following the procedure which may allow for sufficient flow of blood, bone marrow, or both from below the subchondral bone into the defect site.

The offset distance of the guide and thus the spacing between drilled holes will again depend on the bone quality and cellularity of the patient being treated. Smaller, closely-spaced holes are most desired to provide maximum vascularity and maximum attachment of the clot at the defect site and within the bone holes. However, this may not always be possible, particularly when the subchondral bone is weakened, as may be the case in, for example, an osteoarthritic joint. In that situation, holes may be further spaced to compensate for the weaker bone and to avoid collapsing of the subchondral bone from perforation.

In other embodiments of cannulated guides including an offset pin, the guide may have additional offset pins also positioned on the distal tip, such that the at least one additional offset pin may be positioned at another location on the circumference of the distal tip relative to the first offset pin, such that the first and second offset pins are at an angle to one another around the circumference of the distal tip of the guide. For example, a cannulated guide may include a first offset pin on one of a right side, left side, top or bottom, or any angle in between, and a second at an angle to the first offset pin (though the axes of the first and second offset pins are generally parallel to each other, they may, in some embodiments, be transverse to one another). Typically the two pins may be angled at about 90 degrees to one another such that the single guide can properly form a matrix, however other angles may also be used to create other patterns as needed. Forming a matrix of bone holes at a defect site may include the surgeon first using the first guide to form a linear row of bone holes which will be side-by-side to one another. And then, the surgeon may switch to the second guide (such as in FIG. 11) to create a plurality of columns extending from each hole in the original row which will be configured in a top-to-bottom configuration. The at least two pins may both be independently retractable, such that only the pin being used to align a specific bone hole is extended and in use.

In yet another embodiment, the cannulated guides may each include a single pin which is mounted on an offset arm which is configured to rotate about an axis which is substantially parallel to the axis defined by the drill head as it exits the guide. By rotating the offset arm, the surgeon may select the relative angle between two consecutive holes formed using the offset pin. This embodiment may have a further feature that the offset arm may lock at a plurality of defined angles around the circumference of the distal tip. For example the arm may lock in increments of 90°, 60°, 45°, 30°, or 15° of rotation about the circumference of the distal tip. This locking may be accomplished via a spring-loaded ball and socket connection or similar structures commonly used in the art. Such rotation may further allow the cannulated guide to be rotated about its axis, while maintaining the offset pin within an already drilled bone hole, such that proper alignment of the guide, e.g., ensuring the next bone hole to be drilled will be substantially perpendicular to the articular cartilage, can be achieved while still maintaining a proper distance and direction from previously drilled bone holes.

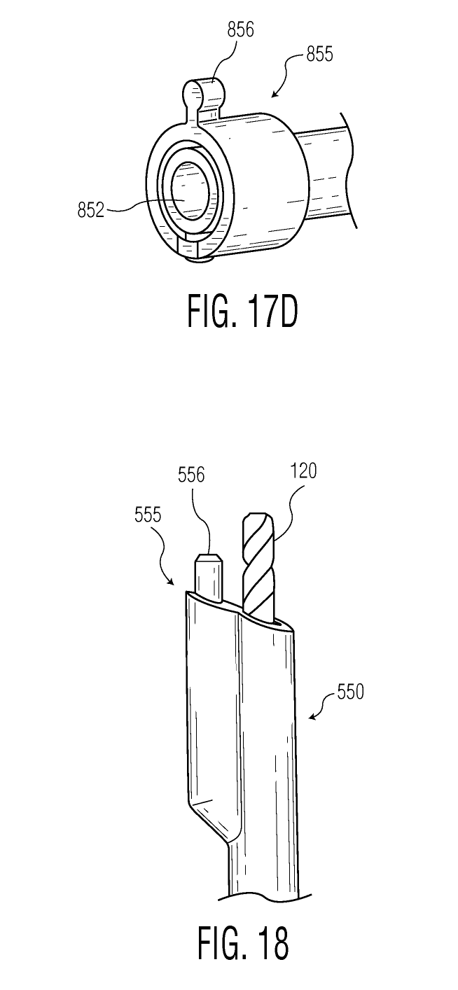

In yet a further embodiment, the cannulated guide 550, 750 of the present invention may have a distal tip 555, 755 having both an offset pin 556, 756 and an angled cut 557, 757, as illustrated in FIGS. 15c (also illustrated in FIG. 18) and 16b. In these examples, the angled cut is A (see FIG. 15c), which is, in this example, 15 degrees. Such a design may be used in areas where the particular anatomy has a slope, such that the angled tip may have a more secure connection to the anatomy than a flat-faced distal tip. Additionally, such an angled tip may be useful where the defect site is difficult to access and thus the distal tip of the guide cannot sit generally perpendicular to the site and sit flush with the tissue. Thus, the angled tip may form a more secure connection to the anatomy and still provide the surgeon with both a secure placement of the guide and an opportunity to form bone holes which are closer to perpendicular to the articular surface than if a flat-faced guide were used.

Of course, as with other embodiments, the angle A of the cut 557, 757 of the distal tip 555, 755 may be other than 15 degrees, and thus the surgeon may have a selection of guides each having a different angle of cut of the distal tip. Additionally, the offset distance may also vary to be a distance other than about 3 mm or about 4 mm, to suit the decision of the surgeon.

In yet another embodiment of the offset pin 856 distal tip 855 of the cannulated guide 850, the distal tip 856 may not be integral with the distal portion of the guide tube 851 and may instead be a removable attachment, as illustrated in FIGS. 17a-d. This embodiment may be useful, for example, where the guides are constructed of stainless steel or the like and are thus autoclavable or otherwise sterilizable. The offset tips may thus be disposable and may be constructed of plastic, or the like, such that they can be easily removed and disposed of following surgery, and the guide may be reused.

FIGS. 17a-d illustrate one embodiment of a removable offset pin 856 which includes a cylindrical receiving portion 858 for sliding over the distal portion of the guide tube 851. The distal portion of the guide tube and cylindrical receiving portion of the offset pin may also have a locking structure 859a, 859b to secure the pin to the distal tip, such as a spring-loaded ball and socket connection, a press-fit, a snap-fit (as illustrated in FIGS. 17a-d), or the like. Additionally, such a configuration may be useful as a kit wherein a single set of cannulated guides, having various angles of curvature, may be coupled to a plurality of removable offset pins having various offset distances, angles of cut, orientations (relative to the direction of curvature of the guide), and the like. FIG. 17d illustrates a cross-section of one embodiment, wherein the selected removable offset pin provides for an offset distance of 4 mm. Other distances may also be provided by adjusting dimensions such as the thickness of the cylindrical receiving portion, the length of the arm connecting the cylindrical receiving portion to the pin, the thickness of the pin itself, or the like. Moreover, other possible receiving portions may be used as well. For example, instead of a cylindrical receiving portion, the receiving portion may only wrap around a portion of the distal tip of the guide and may secure to the guide using a snap-fit. Alternatively, the receiving portion could be one half of a male-female connection and a portion of the surface of the distal tip of the guide may form the other half, such that the male and female portions of the two structures may secure to one another. Other connection structures may also be used as desired.

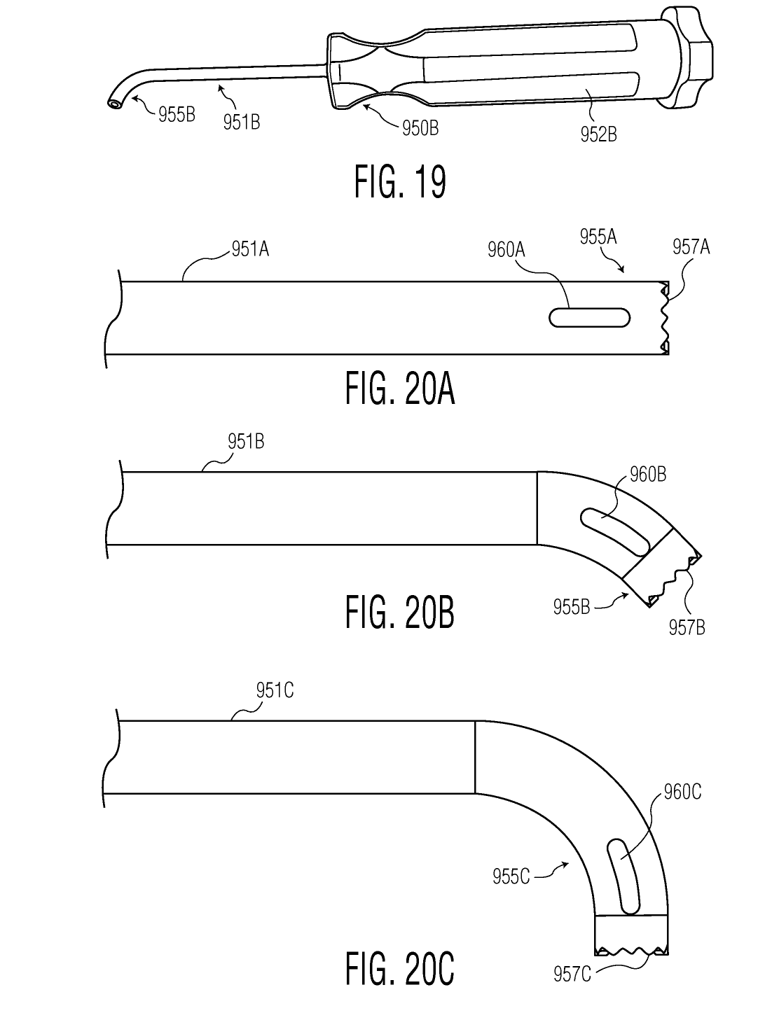

In another embodiment, illustrated in FIGS. 19-20, a cannulated guide 950 includes a handle 952, guide tube 951 and a distal tip 955 (labeled as a, b, and c for the three variations of guide in FIGS. 19-20). As in the various embodiments above, the guide 950 may have a curvature between about 0 degrees and about 90 degrees. For example, FIGS. 20a-c illustrate guide tubes 951a, 951b, 951c having a curvature of 0 degrees (FIG. 20a), 45 degrees (FIG. 20b) and 90 degrees (FIG. 20c). These three variations may form a kit such that the surgeon may select the appropriately curved guide for a specific application, though of course, other guides having other angles of curvature may also be in such a kit.

The distal tip 955, as illustrated in detail in FIGS. 20a-c, may include a window 960, as described above, through which the surgeon may observe the passage of the drill head through the guide. In such a configuration, the flexible shaft may include at least one laser marking (not shown), or the like, positioned circumferentially around the shaft, which may assist the surgeon in determining the depth of the drill head into the defect site and/or subchondral bone.

The distal tip 955 may also include a serrated edge 957 which may improve the engagement of the guide 960 to the bone surface, cartilage or the like at the defect site (similar to the distal-most portion 256 of FIG. 12).

The flexible drill and guides may be reusable and sterilizable, though they may also be disposable after a single use.

The instrument set may further include an instrument (not shown) for implanting a material into the bone hole or plurality of holes drilled into the subchondral bone or to fill the cartilage defect being treated. For example, the material may be a scaffold or matrix, a hydrogel or flowable matrix, cartilage particulates, tissue particulates, a blood preparation, a bone marrow preparation, a cell-based solution, a biological active such as one or a mix of growth factors, or any combination thereof. Further, an adhesive may be used, such as fibrin glue or the like. The material may be from an autologous, allogenic or xenogenic source.

The cartilage may be in the form of powder, fragments, minced tissue, porous forms, sponge-like forms, slurry, hydrogel, or other forms and may be provided fresh-frozen, lyophilized or in another state. Blood preparations may include, but are not limited to, blood components produced using a separation or concentration technique. One example is a platelet concentrate, such as platelet rich plasma, platelet rich fibrin, platelet rich fibrin matrix. Another example is a blood clot. The blood preparation may include platelets, cells, fibrinogen, fibrin, cytokines, proteins, plasma or a combination thereof. Bone marrow preparations may include, but are not limited to, bone marrow components produced using a separation or concentration technique. One example is a bone marrow concentrate, such as BMAC, or a bone marrow clot. The bone marrow preparation may include cells, platelets, fibrinogen, fibrin, cytokines, proteins, plasma or a combination thereof. Alternatively, the cartilage particulates may be pre-mixed with a clotted blood or bone marrow preparation to produce a matrix that can be handled easily and delivered to the repair site. Alternatively, the cartilage particulates or filler is in the form of a matrix, composed of tissue, organic or synthetic materials, which can retain the blood and bone marrow flowing from the subchondral bone holes. The cartilage filler may be a sponge-like, porous form to facilitate cell and clot attachment. The scaffold, matrix, hydrogel or flowable matrix may be composed of polymer, ceramic, collagen, polysaccharide, tissue, extra cellular matrix materials, or a combination thereof.

The drilling of the subchondral bone may allow for bleeding of the subchondral bone, and the bone hole may produce a flow path for the blood and bone marrow to the subchondral bone surface and articular cartilage defect. Blood and bone marrow may in turn clot into the cartilage defect being treated and may provide an environment for cartilage tissue to be regenerated above the area where subchondral bone drilling was conducted. The addition of scaffold or matrix, hydrogel or flowable matrix, cartilage particulates, cartilage filler or matrix, tissue particulates or matrix, a blood preparation, a bone marrow preparation, a cell-based solution, a biological active such as one or a mix of growth factors, or a combination thereof, may provide an enhanced environment and matrix to improve the repair and regeneration of cartilage from the subchondral drilling.

In another embodiment, the present invention may include a method for performing microdrilling surgery including directing a distal portion of a cannulated guide having an angle of curvature adjacent to a defect site; directing a drill, which may include a drill head and a flexible shaft, through the cannulated guide towards the defect site; drilling at least one hole through the defect site and into subchondral bone; and removing the drill and cannulated guide from the defect site and allowing blood or bone marrow to flow into the at least one hole. The method may further include implanting a material into the at least one hole following removal of the flexible drill. The material may pass through the cannulated guide to the hole or may be directed to the defect site through another instrument.

The hole may be drilled such that it is generally perpendicular to the adjacent bone surface, or at any angle the surgeon desires. The method may further include drilling a series of holes into the subchondral bone. Each of these holes may be at the same angle or at a different angle to the other holes. Thus, for example, the surgeon may use a plurality of cannulated guides, each having different angles of curvature, to produce a series of holes having different angles relative to the surface of the adjacent subchondral bone. Also, the same guide may be adjusted to be at different angles relative to the surface of the subchondral bone. Alternatively, the same cannulated guide may be used to prepare the series of holes, while the guide is positioned at the same angle relative to the subchondral bone surface, such that each of the holes is at about the same angle relative to the surface of the subchondral bone such as, for example, generally perpendicular to the surface of the subchondral bone or articulating surface. As discussed above, maintaining the various holes all at a generally perpendicular angle to the surface of the bone is beneficial and is thus preferred.

In yet another embodiment of the present invention, a method of microdrilling surgery may first begin with an arthroscopic evaluation, by the surgeon, at the defect site to determine if microdrilling surgery is a proper procedure for the defect. Of course, the microdrilling procedure may also be done as an open surgery, and thus the surgeon would have an unobstructed view of the defect site prior to starting the surgery. Once the site has been evaluated, the area of the defect may be debrided.

Once the site is thus prepared, a cannulated guide having a desired angle may be placed against the articulating surface, or, if the articular cartilage is completely absent from the area (whether through complete debridement or due to substantial wear and tear on the joint), directly onto the subchondral bone surface. A flexible drill is then passed through the guide (see FIG. 18) until a head of the drill contacts the surface. A hole may then be drilled into the subchondral bone to a desired depth (or a pre-defined depth), and the drill is subsequently removed from the bone. Multiple holes may be prepared using the same process, if desired, until the defect is substantially covered by drill holes. However, care should be taken not to place the holes too close together such that the bone collapses, though the holes should not be spread too far apart such that a continuous, stable clot cannot form across the defect site. For example, the use of an offset pin on the cannulated guide may provide for proper placement of bone holes relative to one another. Alternatively, care should be taken by the surgeon to maintain a proper bone wall between adjacent bone holes.

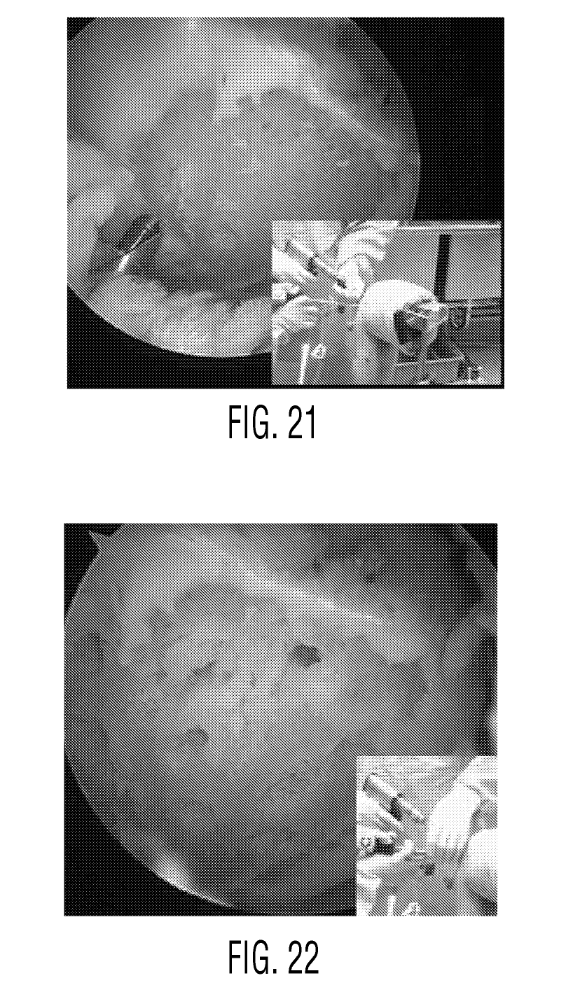

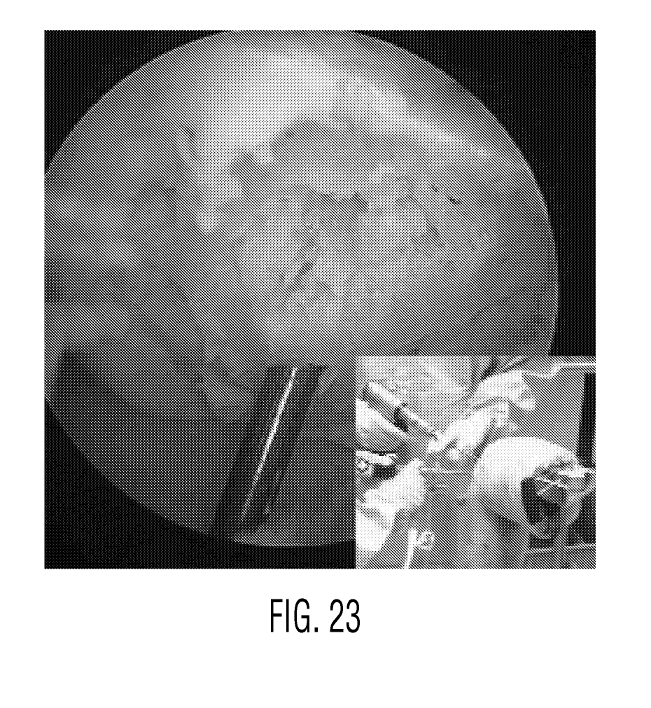

FIGS. 21-23 are photographs of one embodiment of the method of the present invention. FIG. 21 illustrates a flexible drill and a cannulated guide, having an angled cut distal tip, being positioned at an articular cartilage defect in a knee. The distal tip of the guide is positioned against the subchondral bone, and the drill forms a bone hole into the subchondral bone, as in FIGS. 22-23, which may then be repeated to produce multiple bone holes at the tissue defect.

Following preparation of the bone holes, the drill may be removed from the surgical site. An instrument may then be brought to the defect to pack a material within the bone holes (step not shown). The instrument may be passed through the cannulated guide, or alternatively, through another entryway. The material inserted may promote further clotting and regeneration of cartilage, such as, for example, cartilage particulates, cartilage matrix, a blood preparation, a bone marrow preparation, or any combination thereof. Further, an adhesive may be used, such as fibrin glue or the like. The material may be from an autologous, allogenic or xenogenic source and may be implanted in the various forms and compositions as discussed above.

In yet a further embodiment, the present invention may include a kit having at least one flexible drill and at least one cannulated guide. The kit may include multiple flexible drills which may have at least one of, but not limited to, various drill head shapes, various drill head diameters, various depth stop lengths, various flexible shafts having various angles of bend or curvature, various drill head and drill shaft materials or combinations of materials, or any combination of these.

In one embodiment, the cannulated guide may be bendable or flexible to achieve any desired angle but still remain rigid when in use. For this embodiment, a guide made of nitinol may be used which may be bendable at one temperature but rigid at another. Alternatively, the kit may include a plurality of guides, each at a specific angle, such that the kit may include cannulated guides having an angle of curvature from about 0 degrees to about 90 degrees, at various increments such as, for example, 5 degrees, 10 degrees, 15 degrees or the like. For example, the various cannulated guides in the kit may have angles of curvature of 0 degrees, 45 degrees and 90 degrees. Alternatively, the kit may include cannulated guides having an angle of curvature of between greater than 0 degrees up to about 90 degrees. For example, the angle of curvature of the various guides in the kit may be about 15 degrees, about 30 degrees, about 45 degrees, about 60 degrees, and about 90 degrees.

The various cannulated guides may also have an assortment of distal tips, such that the surgeon may select the proper distal tip for the location of the tissue defect. In one example, the cannulated guides may all include offset pins. However, the offset pin on each guide may be positioned on a different side of the guide, relative to the curvature of the guide. Thus, a first guide, having a 60 degree curve, may have a pin located on a right side of the guide, relative to the curve. A second guide, as illustrated in FIG. 11, also having a 60 degree curve, may have a pin located on a top side of the guide, relative to the curve. Then, when forming a matrix of bone holes at a defect site, the surgeon may first use the first guide to form a linear row of bone holes which will be side-by-side to one another. And then, the surgeon may switch to the second guide to create a plurality of columns extending from each hole in the original row which will be configured in a top-to-bottom configuration.

Moreover, as to this embodiment of the kit, the cannulated guides may also have various and differing offset distances of offset pins, angles of cut on the distal tip and/or offset pin, and the like.

Alternatively, the kit may include a plurality of cannulated guides which have two offset pins, a first on one of a right side, left side, top or bottom, or any angle in between, and a second at an angle to the first offset pin and thus at another of a right side, left side, top or bottom which is different from the first pin. Typically the two pins may be angled at about 90 degrees to one another such that the single guide can properly form a matrix, as above, however other angles may also be used to create other patterns as needed. The kit may thus include a plurality of guides, having various angles of curve, each with two offset pins. Of course, any combination of pins (one, two, three, four, and so on offset pins on a guide) on a guide and angles of guides (as previously discussed) may be combined to form a kit.

In yet another embodiment, the kit may include one or more flexible drills, as above, and a plurality of cannulated guides, each having a different angle of curvature from the others. The kit may also include a plurality of removable offset pins, as illustrated in FIGS. 17a-d, which may have various offset distances, angles of cut, positions relative to the angle of curvature of the guides, and the like. The surgeon may thus combine the desired cannulated guide with a desired removable offset tip to best access the defect site and prepare the bone holes. Of course, the surgeon may change either the guide, offset pin, or both, throughout the surgery as dictated by anatomy, progress of surgery, location and size of the defect, and the like.

The kit may further include additional instrumentation for implanting a material within the bone holes, as previously discussed, as well as equipment for preparing the material for insertion.

The drilling of the subchondral bone forms a cleaner bone hole and minimizes any crushing of the porous bone structure. Additionally, the drill of the present invention provides a more efficient, quick and dependable device and method for deeper penetration of the subchondral bone which may provide greater flow of blood and bone marrow from the subchondral bone to the articular surface, and better clotting at the defect site of the articular surface. Additionally, the possible curvature of the flexible drill and cannulated guides allows a surgeon to obtain a better drill angle for difficult to reach defect locations, as well as allow the surgeon to prepare bone holes that are generally perpendicular to the subchondral bone surface and are at a proper distance relative to one another, regardless of the entry angle of the instrumentation into the joint space.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of performing microdrilling surgery comprising directing a distal portion of a cannulated guide having an angle of curvature adjacent to a defect site; directing a drill, including a drill head and a flexible shaft, through the cannulated guide from a proximal portion of the guide through the distal portion and towards the defect site; drilling at least one hole into the defect site; and removing the drill and cannulated guide from the defect site and allowing blood or bone marrow to flow into the at least one hole towards the defect site.

2. The method of claim 1, wherein the defect site comprises subchondral bone.

3. The method of claim 2, wherein the defect site further comprises articular cartilage.

4. The method of claim 3, wherein prior to the step of directing the guide to the defect site, the method further comprises the step of debriding at least a portion of the articular cartilage.

5. The method of claim 4, wherein subchondral bone is exposed.

6. The method of claim 4, wherein calcified cartilage is exposed.

7. The method of claim 2, wherein the at least one hole is generally perpendicular to a surface of the subchondral bone.

8. The method of claim 1, wherein the flexible shaft comprises a spiral configuration including at least one wound coil.

9. The method of claim 8, wherein the spiral configuration of the flexible shaft includes a plurality of wound coils positioned coaxial and concentric with one another.

10. The method of claim 1, wherein the angle of curvature of the cannulated guide is between about 0 degrees to about 90 degrees.

11. The method of claim 10, wherein the angle of curvature is about 0 degrees, about 45 degrees, or about 90 degrees.

12. The method of claim 1, wherein the distal portion of the cannulated guide includes a serrated edge.

13. The method of claim 12, wherein prior to drilling the hole into the defect site, the method further comprises engaging the defect site with the serrated edge.

14. The method of claim 1, wherein two or more holes are drilled into the defect site.

15. The method of claim 1, further comprising the step of packing or injecting a material within the one or more bone hole.

16. The method of claim 15, wherein the material includes cartilage particulates, cartilage matrix, a blood preparation, a bone marrow preparation, or any combination thereof.

17. A method of performing microdrilling surgery comprising:
directing a distal portion of a cannulated guide having an at least one projection at a distal tip and an angle of curvature adjacent to a first location on a defect site;
engaging the defect site with the projection;
directing a drill, including a drill head and a flexible shaft, through the cannulated guide from a proximal portion of the guide through the distal portion and towards the defect site;
drilling a first bone hole into the defect site;
withdrawing the drill from the first bone hole;
disengaging the projection from the defect site; and
removing the drill and cannulated guide from the defect site and allowing blood or bone marrow to flow into the at least one hole towards the defect site.

18. The method of claim 17, wherein, prior to the removing step, the method further comprising:
directing the distal portion of the cannulated guide to a second location on the defect site;
engaging the defect site at the second location with the projection;
drilling a second bone hole into the defect site;
withdrawing the drill from the first bone hole; and
disengaging the projection from the defect site.

19. The method of claim 17, wherein the projection comprises a serrated edge.

20. The method of claim 17, wherein prior to the step of directing the guide to the defect site, the method further comprises the step of debriding at least a portion of the articular cartilage.
